(19) United States
(12) Patent Application Publication
(10) Pub. No.: US 2014/0124374 A1
(43) Pub. Date: May 8, 2014

(54) DEVICE FOR INDICATING THE POSITION AND ORIENTATION OF A DENTAL IMPLANT

(71) Applicant: Nobel Biocare Services AG, Zurich-Flughafen (CH)

(72) Inventors: Thomas Eriksson, Goteborg (SE); Magnus Ottsjö, Vastra Frolunda (SE)

(73) Assignee: Nobel Biocare Services AG, Zurich-Flughafen (CH)

(21) Appl. No.: 14/069,224

(22) Filed: Oct. 31, 2013

Related U.S. Application Data
(63) Continuation of application No. 13/202,761, filed on Sep. 13, 2011.
(60) Provisional application No. 61/155,675, filed on Feb. 26, 2009.

(51) Int. Cl. A61C 1/08; A61B 5/00

(52) U.S. Cl. CPC A61C 1/084; A61B 5/0088
USPC 205/112; 205/333; 205/322; 205/221

(57) ABSTRACT

A position locator for use in dental restorative procedure is described. The position locator is inserted into a replica of a dental implant or into a replica of an abutment. The position and orientation of the implant replica is determined by scanning the model with the implant replica and the position locator. Alternatively, the position locator can be inserted into the dental implant and scanning is carried out in the mouth of the patient. The position locator is made of an optically opaque material, such as titanium, and has an outer surface detectable by an optical scanner, e.g. with a layer of porous titanium oxide applied through anodic oxidation.

### PRIORITY INFORMATION

This application is continuation of U.S. patent application Ser. No. 13/202,761, filed Sep. 13, 2011, which is a U.S. National Phase of International Application No. PCT/EP2010/001153, filed on Feb. 25, 2010, which claims priority to U.S. Provisional Application No. 61/155,675, filed Feb. 26, 2009. The entirety of PCT/EP2010/001153 is hereby incorporated by reference.

### BACKGROUND

1. Field of the Inventions

This disclosure of the present inventions pertains in general to the field of oral, dental or maxillofacial restorative medical procedures, and products related thereto. More particularly, the disclosure relates to devices and methods for facilitating determination of a position and orientation of components, like surgical implants, involved in such procedures. Even more particularly, the disclosure relates to position locators affixable to said components in a defined relationship, having at least one surface detectable by optical scanning for the position and orientation determination.

2. Description of the Related Art

An optical scanning method and apparatus is for instance disclosed in U.S. Pat. No. 6,590,654, which is incorporated herein by reference in its entirety for all purposes.

There is a need for providing position locating devices for detection by such optical scanning apparatuses, which are advantageous from a point of view of detectability with high precision. These position locating devices are hereinafter also referred to as position locators.

An application where such position locators are needed is the field of dental restorative procedures.

### SUMMARY

Accordingly, some embodiments of the present inventions preferably seek to mitigate, alleviate or eliminate one or more deficiencies, disadvantages or issues in the art, such as the above-identified, singly or in any combination by providing a position locator device, a system, a method, and a computer program product according to the appended patent claims.

According to a first aspect of some embodiments, a position locator device is provided. The position locator device is provided for a system of planning and producing oral or maxillofacial restorative products. The device is made of an optically opaque material, and has at least one outer surface that is detectable by an optical scanner apparatus for determining at least one of a position and orientation of the outer surface. The device further has a layer deposited onto the outer surface of said position locator.

In some embodiments, the outer surface is optically more diffusive reflective than specularly reflective.

In some embodiments, the outer surface is a metal oxide layer deposited onto a body of the position locator device by anodic oxidation.

In some embodiments, the metal oxide layer has a homogenous layer thickness.

In some embodiments, the layer has a substantially uniform mean thickness.

In some embodiments, the material is a metal.

In some embodiments, the metal is titanium.

In some embodiments, the outer surface is a porous titanium oxide layer deposited onto a body of the position locator device by anodic oxidation.

According to a second aspect of some embodiments, a kit of at least one dental restorative product and at least one position locator as mentioned above can be provided.

In some embodiments, the dental restorative product is an implant replica or an abutment replica.

According to another aspect of some embodiments, a method is provided. The method is a method of improving precision in an optical scanning method, comprising providing a position locator as mentioned above for said optical scanning method.

In some embodiments, the optical scanning method is conoscopic holographic scanning.

Further details of some embodiments are defined in the dependent claims, wherein features for the second and subsequent aspects of some embodiments are as for the first aspect mutatis mutandis.

Some embodiments provide for improved precision of determination of a position and orientation of a dental implant by optical scanning.

Some embodiments also provide for providing input data with improved precision for virtually planning a dental restoration, and thus production data for dental restorations can be provided with improved precision.

Some embodiments provide improved detectability of position locators by optical scanning in combination with improved precision of the dimensions of the position locators within desired tolerances for dental restorative procedures.

Some embodiments also provide for improved matching or merging of a plurality of data sets thanks to improved resolution of data provided by optical scanning of improved position locators. Some embodiments provide for improved merging of patient data and data for a dental restoration.

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which embodiments of the inventions are capable of will be apparent and elucidated from the following description of embodiments of the present inventions, reference being made to the accompanying drawings, in which

FIG. 1 is a flow chart of a method;

FIGS. 2A-E are various views of an embodiment of a position locator;

FIGS. 3 and 4 are perspective views of a plurality of position locators affixed to a model of a patient's jaw; and

FIGS. 5A-D are various views of another embodiment of a position locator.

### DESCRIPTION OF EMBODIMENTS

Specific embodiments of the inventions will now be described with reference to the accompanying drawings. These inventions may, however, be embodied in many different forms and should not be construed as limited to the embodiments set forth herein; rather, these embodiments are provided so that this disclosure will be thorough and complete, and will fully convey the scope of the inventions to those skilled in the art. The terminology used in the detailed description of the embodiments illustrated in the accompanying drawings is not intended to be limiting of the inventions. In the drawings, like numbers refer to like elements.

The following description focuses on an embodiment of the present inventions applicable to a dental restorative procedure. However, it will be appreciated that the inventions are not limited to this application but may be applied to many other surgical procedures, including for example maxillofacial restorative surgical procedures.

### Position Locators

In embodiments, a position locator device for a system of planning and producing oral or maxillofacial restorative products, is provided. The position locator device is made of an optically opaque material, and has at least one outer surface detectable by an optical scanner apparatus for determining a position and orientation of said outer surface. In this manner, a position and orientation of the position locator device is determinable.

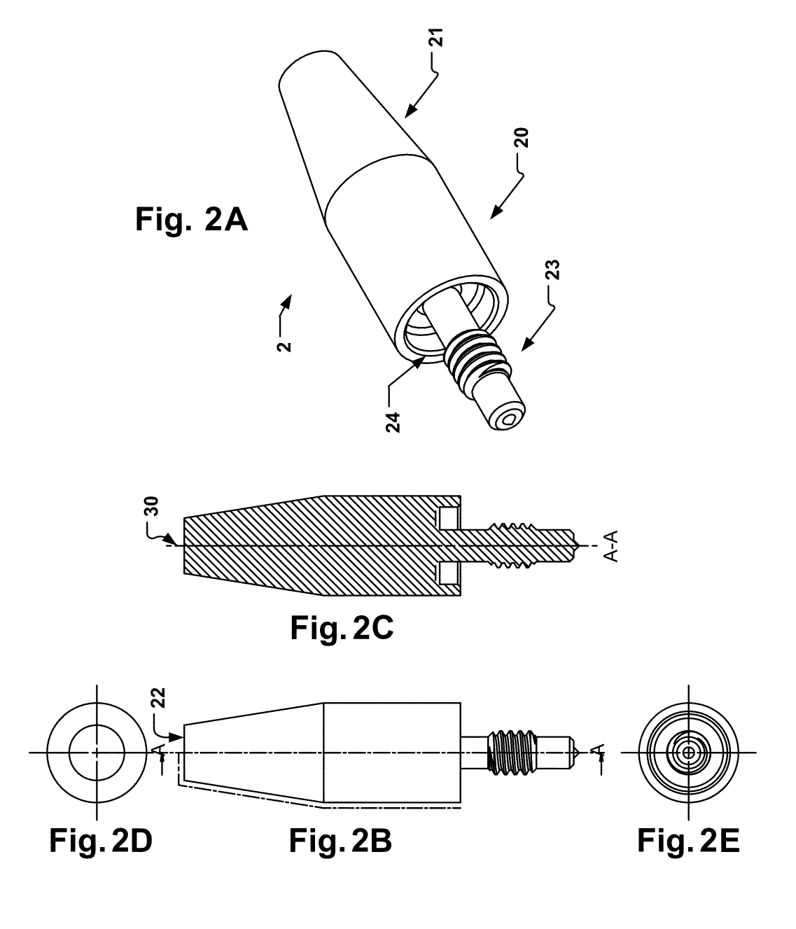

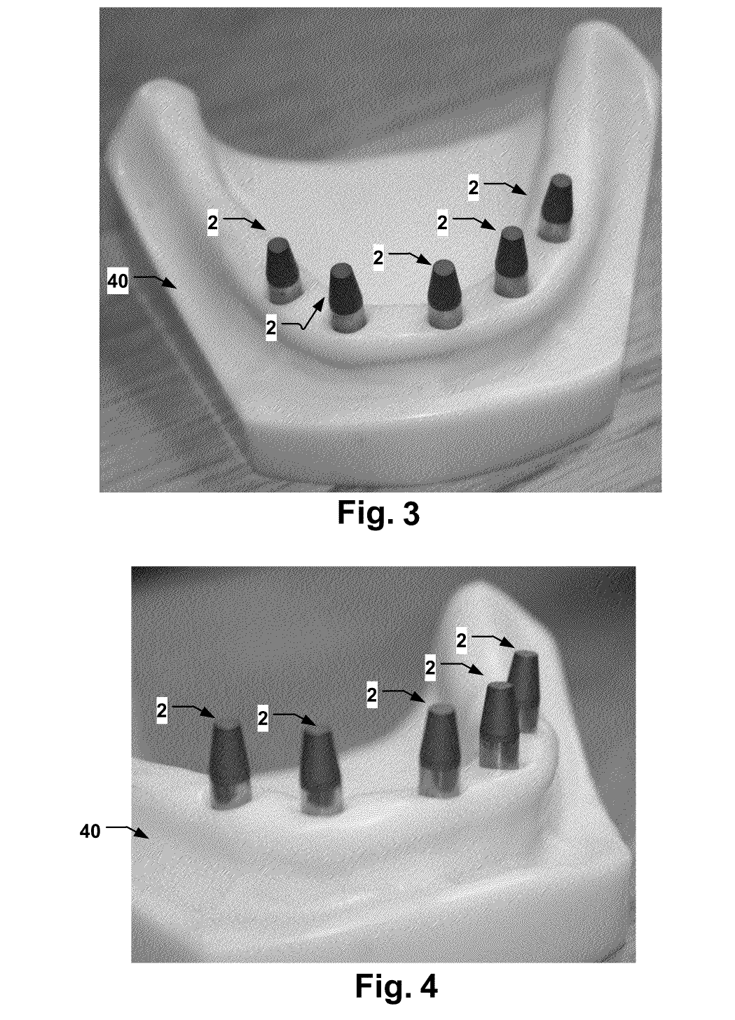

An embodiment of a position locator 2 is illustrated in FIGS. 2A-E. In FIGS. 3 and 4 such a position locators are illustrated inserted into dental implant replicas in a model 40 of a dental situation of a patient prior to providing a final dental restoration.

The position locator has a cylindrical portion 20, a conical portion 21, a top portion 22, a threaded portion 23 and a connection interface 24.

At least a portion of the position locator surface is dull for improved optical scanning and measurement of positions thereof, as is described further below. The diffuse reflection provided by the surface provides high precision when optically scanned.

In some embodiments, the position locator body is made of titanium. The surface is an oxidized surface of the material. The oxidized surface has a layer thickness. The oxidized surface has a surface roughness. The geometrical dimensions of the position locator are provided with very high precision, as will be explained below.

The conical portion and the top portion provide a frusto conical portion which longitudinal center axis coincides with a longitudinal center axis 30 of the position locator device 2.

The conical portion 21 is used for calculation of the longitudinal center axis 30. The top surface 22 of the frusto conical portion is used to determine the position in space of the position locator 2. In this manner, a vector for the position and orientation of the position locator 2 is determinable by optical scanning. When the position locator 2 is affixed to another structure having a mating connection interface, such as a dental implant, it has a precisely defined geometrical relation and position in relation to the other structure. The position locator 2 is affixed to another structure by means of the helically threaded portion 23. By registering the position locator 2, the position of the other structure can be determined as there is a defined relation between the two.

The position locators may be rotationally symmetrical. Rotationally symmetry may be used in applications where the rotational orientation of the position locator is not needed or not of importance. This is, for instance, the case when the rotation of a dental implant around the longitudinal axis thereof is irrelevant. This is for instance the case when at least two implants are used to bear a connecting structure providing rotational locking itself. It is sufficient to use rotationally symmetrical position locators. For instance, dental bridges are fixated to a jaw of a patient by using at least two dental implants. Here, the position and longitudinal orientation of the implants are determined by using rotationally symmetrical position locators, such as illustrated in FIGS. 3 and 4.

Rotationally non-symmetrical position locators are provided in some embodiments (not shown). Non-symmetry may be provided by non-symmetric top surfaces, or lateralplanar surfaces, e.g. along a conical portion of the position locator.

The position locators may have different diameters.

The position locators may have different mating interfaces for connection interfaces of components to which they are releasably attachable.

Embodiments of the position locators are made of titanium. Titanium is an advantageous material for a body of position locators because a titanium oxide layer can be provided on a surface thereon that is particularly optically advantageous, as is described below.

The position locators are for instance produced by turning and/or milling from a raw material. The manufacturing method applied depends on various factors, including the shaping of the position locator, the connection interface thereof, etc. Rotationally symmetrical position locators may be manufactured by turning only. Rotationally symmetrical position locators may be manufactured partly by turning a raw material into a rotationally symmetrical intermediate product. The intermediate product is subsequently machined to comprise at least one non-rotationally symmetrical surface. This is for instance done by milling or cutting, whereby material is removed from the intermediate product. Thereby a surface is formed that has a defined relation to a rotational position in longitudinal direction of an implant when the position locator is affixed thereto. Such a position locator providing a rotational registration may be used for a dental implant for a single tooth, where the rotational locking capability of the implant is of importance.

By frequently used machining steps, such as milling or turning, of a metal, and in particular titanium, the machined surface of the positioning locator body becomes very smooth and glossy. Such machined surfaces have proven to be less well suited for detection by optical scanners, such as described in U.S. Pat. No. 6,590,654. In particular, U.S. Pat. No. 6,590,654 refers to conoscopic holographic scanning. Scanning position locators having machined surfaces results in erroneous measurements. Instable measurement values are obtained. This is due to the optical properties of the machined surfaces. The machined surfaces are too glossy. Incident light from the scanner light source is mostly reflected and does not reach the optical detector, which is oriented in the same direction as the incident light beam. When the incident light beam is directed non-perpendicularly onto a machined surface, it is reflected away. In this manner, a too low or no backscattered light reaches the detector and a position and orientation measurement cannot be provided correctly. For instance, a position and orientation of a cylindrical, partly frusto conical position locator is not determined sufficiently well for providing data suitable for producing components to be used in dental restoration procedures. The measurement result has to lie within tolerances of as low as in the range of a few micrometers. This is in particular of importance, where a plurality of dental implants are used for affixing a single structure, like a dental bridge, to a jaw. In case the dental bridge framework is produced from erroneous data, it will not fit the implants installed in the patient. Also for single tooth restorations, such narrow tolerances are needed in order to provide dental restorations that fit in relation to surrounding teeth. In case a single tooth restoration is produced from erroneous data, it will not fit into the space provided by the surrounding teeth. This is costly and cumbersome for the patient and has to be avoided.

Thus, a finishing process to modify the machined surfaces to be less glossy is applied. The surface is made optically dull, such that diffuse scattering of incident light is predominant. However, as the position locator has to be provided with dimensions having very low deviation from a desired production value, this finishing process has to be carefully applied. It is desired that the finishing process as little as possible influences the physical dimension of the position locator.

Having in mind, that tolerances of as low as in the range of a few micrometers, are required, this is a demanding task. Many finishing procedures provide modified machined surfaces that are not suitable for this purpose. Finishing procedures may be based on removing material from the machined surface, or depositing layers of material onto the machined surface. However, most finishing procedures provide an inhomogeneous surface modification, which is not acceptable in the present case.

For instance, abrasive blasting with sand, for example, is not suited for this purpose. Sandblasting is made with a jet stream of sand grains. This jet stream is inhomogeneous in itself due to the distribution of sand grains therein. Furthermore, overlapping treatment occurs by passing with the jet stream over the surface to be treated. Moreover, such sandblasting abrasion leads to a removal of more material than allowed within the presently desired tolerances. In this manner, it is impossible to control the finishing process to provide a homogenously finished surface within the desired tolerances.

Many techniques for creating layers having less glossy optical properties are not suited for the present purpose for similar reasons. For instance, chemical vapor deposition (CVD) of a material onto a machined surface does not provide a sufficiently homogenous layer thickness. Variations of the layer thickness are larger than the allowed tolerances.

In this manner, although a sandblasted surface or a CVD treated surface has improved optical properties increasing the diffuse scattering, and thus may provide considerably better input signals to the optical scanning detector, the resulting measurements of such position locator would not be suited for providing data for production of dental restorative components. Such position locators provide input data being outside of the allowed tolerances. Hence, production data based on such input data and products manufactured on such production data are not suited for demanding applications such as dental restorative procedures and products.

A surface treatment process for producing an oxide layer on a metallic object is disclosed in WO 00/72776 of the same applicant as the present application, which hereby is incorporated herein by reference in its entirety for all purposes. The surface treatment process is described in WO 00/72776 with reference to implants for bone or tissue structures. An application of the method with position locators is not disclosed. Further, the purpose of the process described in WO 00/72776 is to provide a porous surface layer on an implant that has advantageous osseointegration properties.

In the context of the present application, the process described in WO 00/72776 is used in a novel manner. The process is used to provide improved optical properties of a surface. The process is applied on a position locator. In more detail, the process comprises anodic oxidation and is applied to position locators for providing an advantageously homogenous dull surface having improved precision of detectability by optical scanning.

The anodic oxidation process provides a very homogenous layer of oxide within very narrow tolerances. The process is advantageously controllable to provide a thin layer of oxide that lies within the desired tolerances, but considerably improves the optical properties of the surface with regard to optical scanning of the surface. In this manner, the final dimension of the position locator with the finished surface is determinable with high precision. The position locator is machined to a dimension which has the oxide layer thickness subtracted. When the oxide layer is added homogenously, the final product is provided to a desired dimension within very narrow tolerances, as required for dental restorative procedures and related products. Furthermore, the surface is advantageous for optical scanning, thus providing a very high precision of detection of a position and orientation of the position locator and related structures thereto.

Tests made by the applicant show that optical scanning of position locators having such modified surface provide a very good registration of the position and orientation of the position locator. Signals provided were very good, and considerably improved compared to position locators having machined surfaces.

The oxide layer is preferably made thinner than an oxide layer on implants. The layer thickness provided on position locators is in the range up to a few μm, such as up to 5 μm.

For providing the anodically oxidized surface, the position locator is positioned in an acid. Embodiments of specific acids are described in WO 00/72776, which was previously incorporated herein by reference for all purposes. The anodic oxidation process is performed with a constant current and a voltage that is ramped up to a maximum voltage. The maximum voltage determines the layer thickness of the oxide layer and the process automatically ceases when the maximum voltage is obtained. The layer thickness obtained by the anodic oxidation process is thus controlled in a very precise manner. The maximum voltage is chosen lower than a maximum voltage that would be chosen for generating oxide layers on implants. A typical maximum voltage is in the range of 200-220 Volts.

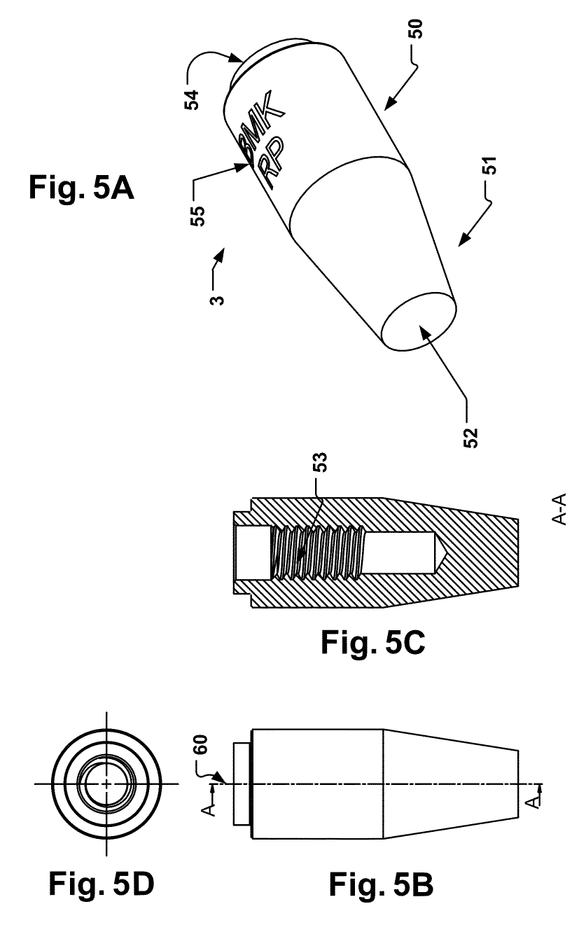

Furthermore, the anodic oxidation process allows for a controlled geometric coverage of surfaces of position locators. The position locator may either only be submerged partly into acid during the anodic oxidation. Alternatively, or in addition, the position locator may be masked suitably to only allow oxidation of selected surfaces. The position locators may thus, for instance, be provided with surfaces that are not optically dull. The non-treated surfaces may be provided with product markings, color coding, etc. such as illustrated in FIG. 5A at markings 55.

In another embodiment according to FIGS. 5A-D, a position locator 3 is provided. The position locator 3 is a bridge position locator for attachment to a model of a dental restoration, like a model of a bridge framework. Thanks to the position locator, the position of the connection interface of a model of a dental restoration is determinable. This determined position of the connection interface is used in the method described below. The position locator 3 has a cylindrical portion 50, a conical portion 51, a top portion 52, an internally threaded portion 53 and a connection interface 54 for matingly engaging a dental restoration.

### Optical Effects of a Surface Having an Oxide Layer

When an incident light ray is reflected from surfaces of optically opaque surfaces, the reflectance has several components, depending on the properties of the surface, such as roughness thereof. Three components of reflected light usually occur, namely a specular reflection, a specular lobe and a diffuse lobe.

For a metallic mirrored surface, the incident light will be reflected by the surface following the law of reflection. In this case, the specular lobe is almost zero, the diffuse lobe is negligible, and the specular spike is very narrow and the intensity substantially corresponds to that of the incident light. In this case no backscattering occurs, and an optical detector arranged in direction of the incident ray will not detect a signal. Therefore, metallic mirrored surfaces, like polished surface are not suitable for optical scanning.

For glossy or smooth surfaces, like the aforementioned machined metallic surfaces, the situation is similar. An incident light ray will be reflected by the surface following the law of reflection. In this case, the specular lobe is larger than in the case of a mirrored surface, but directed mainly in the reflected direction; the diffuse lobe is larger, but negligible in the direction of the incident light and directed mainly in the reflected direction; and the specular spike is somewhat broader than in the previous case of mirrored surfaces and has lost some intensity to the aforementioned two lobes. Still, very little light is backscattered. Therefore, machined metallic surfaces, like turned or milled surfaces are not well suited for optical scanning.

For dull surfaces, like the aforementioned anodically oxidized surface, the situation is different. Here, the specular lobe is larger than in the case of the glossy or smooth surfaces, and much broader, deviating from the reflected direction; the diffuse lobe is largest and most intense, as well as a considerable portion of the incident light intensity is scattered back into the direction of the incident light; and the specular spike is almost nonexistent or not at all existent. Therefore, the surface is optically more diffusive reflective than specularly reflective, and provides a portion of backscattered light that makes the dull surface well suited for detection by optical scanning with high precision.

The position locators of embodiments are provided with at least a surface of the latter category. The portion of light detectable from such surfaces when illuminated is improved considerably as the portion of the diffuse lobe in relation to the entire reflected light is dominant. This is in particular advantageous for optical scanning methods as described in U.S. Pat. No. 6,590,654.

### Examples of Use of Position Locators

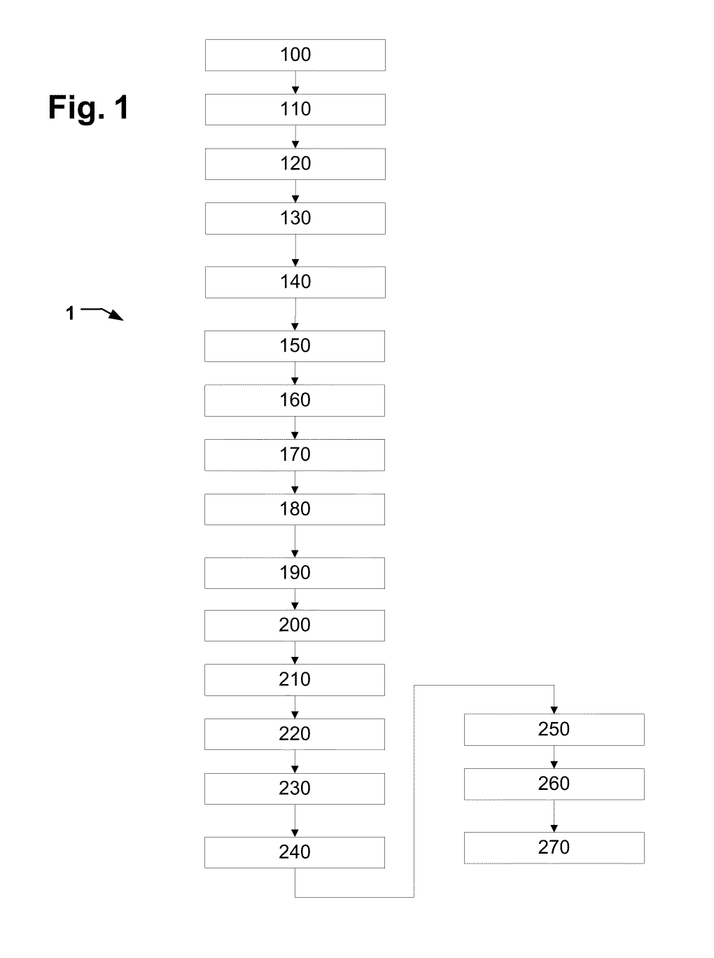

FIG. 1 is a flow chart of a method for preparing data and producing a dental restoration, using position locators during optical scanning.

In an initial point 100 of the method 1, an implant is provided implanted in bone tissue of a patient. After a healing and osseointegration period a component, such as a dental restoration, like a single tooth, a bridge, or other framework, have to be produced and affixed to the implant at a connection interface thereof. For this purpose, the exact position and orientation of the implant have to be determined.

To this end, an impression of the dental situation is taken in step 120.

The position and orientation of an implant or an abutment in a model, or an impression coping in an impression has to be determined with high precision by optical scanning methods.

The impression is a negative of at least a portion of the oral cavity of a patient. The impression is made of a jaw portion comprising an already implanted dental implant. The implant has been implanted previously in the jaw bone tissue, and was optionally left in place for healing and osseointegration with the jaw bone tissue for a solid fixation therein. Thus, the dental implant provides an anchored platform having a defined connection interface for fixation of dental restorations to the jaw bone. Dental restorations comprise dental bridges, single tooth restorations, etc. The dental restorations may be directly fixated to the dental implant or intermediate components like abutments may be used.

From the impression, data will be derived for the position and orientation of the dental implant. A plurality of dental implants may be present. As the implants are anchored in jaw bone tissue, only a top portion of the connection interface or abutment protruding into the oral cavity would be present in impressions. In order to provide improved orientation data for the longitudinal axis of the dental implant, impression copings and position locators are used during impression taking and subsequent scanning of the impression or a model prepared from the impression, respectively. This will now be explained in more detail.

The impression is taken from the patient with impression copings, which are attached to the dental implant(s) in step 110. This may be done by threadably insertion, snap fit, friction fit, or the like. The impression copings thus leave a negative impression in the impression, or are left in the impression when the impression material has solidified. The impression is then removed from the patient in step 130. As the impression copings are fixated to the dental implants, these are provided with a smooth surface such that they easily loosened from the solidified impression material. The impression copings are removed from the dental implants in step 140. The impression and the impression copings are then available for further processing steps towards producing a dental restoration.

An implant replica or an abutment replica is attached to the impression coping in step 150. This assembly is repositioned in the impression into the corresponding recess in the impression in step 160. In this manner, the implant replica or the abutment replica protrudes from the impression at the exact position and in the precise orientation relative to the registered anatomical situation of the patient from which the impression was taken.

The position locator may also be used for intraoral scanning, wherein the position locator is attached directly to the implant in the oral cavity of the patient. In particular, the intraoral scanning is an optical scanning. The optical scanning is performed by means of an intraoral optical scanner.

A model is then cast from the impression in step 170. When the model is solidified, it is removed from the impression, together with the impression coping(s) in step 180. This model corresponds with high precision to the anatomical situation of the patient. Implant replica in the model are precisely positioned at correct positions and with correct orientation in the model. The impression coping(s) are then threadably removed from the fixated implant replica or abutment replica in step 190. As impression coping have a smooth surface these are not suited for optical scanning.

From the model, a dental restoration model, such as a bridge framework model is prepared in step 200, manually taking into consideration aesthetic, geometrical, and mechanical requirements. The bridge framework model may be, for instance, an acrylic framework prepared by using non-engaging temporary abutments or cylinders. The acrylic framework may then be manually reduced to a desired shape.

Subsequently, the model and the dental restoration model are scanned with an optical scanner to provide patient data and data for the dental restoration in steps 210, 220, respectively. The patient data and data for the dental restoration are subsequently matched and provided in a virtual computer based environment in step 230 for planning the final restoration in step 240. Merging of the patient data and data for the dental restoration is facilitated thanks to the use of the improved position locators providing data with higher precision and resolution. When virtual planning is finished, production data is provided in step 250 for manufacturing of a dental restoration, such as a bridge framework, to be installed in the patient. The production is illustrated as step 260.

Prior to scanning, the position locators are threadably affixed to the implant replica or abutment replica. The position locators are now arranged in a precise relationship to the implant replica or abutment replica, corresponding to the anatomical situation of the patient. By detecting the position and orientation of the position locators from optical scan data, the exact position of the implant replica or abutment replica is determinable. This is very important for being able to provide high precision production data and final dental restorations based on this production data.

Prior to scanning, position locators are also threadably affixed to the dental restoration model. These position locators provide, when scanned in an optical scanner, exact data for the position of a connection interface mating with a connection interface of the replica implants or replica abutments.

A plurality of dental implants or abutments may be present. In this case, one position locator will be used for every dental implant.

From optical scanning, the model and the dental restoration model are digitized to provide corresponding data. A calculation is made where the implants or copings are located. As the position locators provide high precision data from optical scanning, this is facilitated by the position locators. For instance, the conical portion of the frusto conical portion is used for calculation of the longitudinal center axis of a position locator. The top surface of the frusto conical portion of the position locator is used to determine the position in space of the position locator. In this manner, a vector for the position and orientation of the position locator is determined. As the longitudinal axis of the position locator and the dental implant or abutment coincide or have a defined relationship, the orientation of the dental implant or abutment is determinable with high precision. As the top portion is provided with high precision and has a defined relation to the connection interface, also the exact position of the dental implant or abutment is determinable.

Also, the dental restoration model having position locator(s) affixed thereto provides data for the position of the connection interface mating with the connection interface of the dental implants or abutments with high precision. Position locators for the dental restoration model, which for instance are shown in FIGS. 5A-D, differ from position locators for implants or abutments, which for instance are shown in FIGS. 2A-E, with regard to the connection interface. The connection interfaces of the dental restoration model and the implants or abutments are mating male/female connection interfaces. Consequently, the connection interfaces are matingly arranged female/male connection interfaces.

The connection interfaces themselves differ in dependence of the specific implant system used.

The patient data and data for the dental restoration are subsequently matched and provided in a virtual computer based environment for planning the final restoration.

When virtual planning is finished, production data is provided for manufacturing of a dental restoration, such as a bridge framework, to be installed in the patient. Further adaptation of the manufactured product, e.g. veneering may be performed manually before finally installing the dental restoration in the patient. Installation is performed smoothly in step 270 as the dental restoration perfectly fits to the dental implants already installed in the patient.

Although embodiments of these inventions have been disclosed in the context of certain examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. Different method steps than those described above, performing the method by hardware or software, may be provided within the scope of the invention. The different features and steps of the invention may be combined in other combinations than those described. It is also contemplated that various combinations or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions.

1. A method of manufacturing a position locator device for a system of planning and producing oral or maxillofacial restorative products, said device being of an optically opaque material, and having at least one outer surface detectable by an optical scanner apparatus for determining at least one of a position and orientation of said outer surface, which method comprises:
providing a metal oxide layer, by anodic oxidation, onto said position locator device to form said outer surface of said position locator device.

2. (canceled)

3. The method according to claim 1, wherein said metal oxide layer has a substantially homogenous layer thickness.

4. The method according to claim 1, wherein said optically opaque material is a metal.

5. The method according to claim 4, wherein said metal is titanium.

6. The method according to claim 5, wherein said outer surface is a porous titanium oxide layer.

7. The method according to claim 1, wherein said outer surface is optically more diffusive reflective than specularly reflective.

8. (canceled)

9. (canceled)

10. (canceled)

11. (canceled)

12. (canceled)

13. (canceled)

14. (canceled)

15. (canceled)

16. (canceled)

17. (canceled)

18. The method according to claim 1, wherein said step of providing a metal oxide layer comprises providing a metal oxide layer having a thickness of up to 5 μm.

19. The method according to claim 1, further comprising the step of machining the position locator to a dimension which has a thickness of said metal oxide layer subtracted.
